(19) United States
(12) Patent Application Publication
(10) Pub. No.: US 2004/0172666 A1
(43) Pub. Date: Sep. 2, 2004

(54) Transgenic birds and method of producing protein using same

(76) Inventors: Shinji Iijima, Nagoya-shi (JP); Masamichi Kamihira, Nagoya-shi (JP); Kenichi Nishijima, Nagoya-shi (JP); Kenichiro Ono, Ina-shi (JP); Kazuhisa Yamaguchi, Komaki-shi (JP); Shinji Mizuarai, Tsukuba-shi (JP)

Correspondence Address: KENYON & KENYON, 1500 K STREET, N.W., SUITE 700, WASHINGTON, DC, 20005, US

(21) Appl. No.: 10/641,001

(22) Filed: Aug. 15, 2003

(30) Foreign Application Priority Data
Feb. 7, 2003 (JP) 2003-031634

(51) Int. Cl. A01K 67/027; C12N 15/867

(52) U.S. Cl. 800/19; 435/456; 119/300

(57) ABSTRACT

The invention has a object to provide a G0 transgenic chimera bird which can efficiently accumulate the useful protein in eggs of a female bird in which the protein is expressed in an oviduct-specific manner and can accumulate even a toxic protein in eggs with no ill effect for birds, and a transgenic bird and a method of producing them. The above object can be attained by providing a G0 transgenic chimera bird and transgenic bird obtained by introducing a replication-defective retrovirus vector coding for a useful protein by an oviduct-specific expression promoter.

### TECHNICAL FIELD

The present invention relates to oviduct-specific gene expression in a transgenic female bird for efficient accumulation of a useful protein in her eggs. It also relates to a vector for use in producing transgenic birds capable of efficiently transmitting such character to their offspring, and a method of producing them.

### BACKGROUND ART

While a number of pharmaceutical protein preparations have been developed, erythropoietin (EPO) and like proteins requiring a sugar chain(s) for their activity manifestation cannot be produced in such inexpensive production systems as Escherichia coli but, for the time being, they can be produced only in animal cells which increase the production cost. It is transgenic animals that have currently attracted attention from the viewpoint of low cost production of sugar chain-bearing proteins.

Since the first appearance of transgenic mice in the world, a large number of transgenic animals have been produced. In recent years, attempts using the above technology have been made in various places of the world to create transgenic cows and swine, among others, and cause them to produce various useful proteins. They are the so-called animal factories.

Actually, sheep secreting human α-antitrypsin into milk have already been produced in Great Britain. However, problems are encountered with large-sized mammals; for example, their growth is slow and large spaces are required for their raising, hence time and cost are required for commercial production of useful proteins.

Poultries, typically quails and chickens, have long been bred for meat and for eggs they lay. As compared with large-sized mammals, they have advantages; their growth rate is high and only small spaces are required for their raising, hence it is possible to produce useful proteins at low cost.

The method generally employed in producing transgenic animals comprises microinjecting DNA into the pronucleus of a fertile egg. However, this method cannot be applied to birds. For obtaining an embryo of a bird at the unicellular stage, the female bird must be killed, and it is difficult to identify the nucleus in the egg. Accordingly, the technology of producing transgenic birds was behind as compared with other mammals.

Recently, however, the transgenic bird technology has also been advancing, and several cases have been reported of the production of transgenic chimera birds by the methods which are other than the conventional technologies of producing transgenic animals but use viruses. There is also an example in which β-galactosidase expression was attempted using part of the ovalbumin promoter (Patent Document 1: Japanese Kohyo Publication 2000-512149).

The expression promoters used in these reported transgenic birds are mostly expressed in the whole body of each bird and, in this case, the desired useful protein is recovered from a small amount of blood or is recovered from tissues or blood after killing the bird. This procedure requires much work and time, hence is inexpedient. For efficient recovery of a useful protein, it is essential for producing the useful protein by causing the same to be accumulated in eggs and recovering the same. One method therefore comprises recovering the protein expressed in the whole body and accumulated in eggs after circulation in blood. In the case of Fc portion-containing proteins, such as antibodies, the proteins can be accumulated in eggs and recovered efficiently by that method since Fc has a characteristic such that it migrates to the yolk.

However, the application of this method is restricted to Fc portion-containing proteins, such as antibodies, and the method has a problem in that the accumulation of other useful proteins in eggs is not always high even when the level of protein expression is high in the whole body. Another drawback is that this method cannot be employed for those proteins which are highly toxic to birds.

In the example (Patent Document 1) showing an attempt at β-galactosidase expression using part of the ovalbumin promoter specifically expressed in the oviduct, which is an egg-producing organ, there is something lacking concreteness and that β-galactosidase is actually expressed in an oviduct-specific manner is still doubtful.

### SUMMARY OF THE INVENTION

To overcome the problem of how to accumulate an Fc portion-free useful protein efficiently in eggs, the present inventors paid attention to egg white proteins. The egg white comprises about 30 or more proteins, among which main ones are ovalbumin (54%), ovotransferrin (or conalbumin; 12%), ovomucoid (11%), ovoglobulin (8%) and lysozyme (3.5%). The promoters for these proteins are expressed specifically in the oviduct and cause accumulation in egg whites of the proteins expressed under the control thereof. They considered that the use of the promoters for those proteins as expression promoters for useful proteins might lead to efficient accumulation of useful proteins in eggs.

Thus, the present inventors used, as a vector, Moloney murine leukemia virus, which is a replication-defective retrovirus vector used in producing transgenic birds. Using that region of the ovalbumin promoter which causes oviduct-specific expression, they constructed a plasmid coding for a viral vector expressing human growth hormone, which is useful as a medicine, and then produced packaging cells for the production of that viral vector. Using the packaging cells, they produced the viral vector, and produced G0 transgenic chimera birds by microinjection of the viral vector into quail and chicken embryos just after oviposition. The gene introduced into the genomes of these G0 transgenic chimera birds was identified by the PCR method. The G0 transgenic chimera quails produced were mated with nontransgenic quails, and the G1 transgenic female quails obtained were subjected, after sexual maturation, to analyses. RT-PCR was carried out for checking the expression of human growth hormone, and it was found that the human growth hormone expression was oviduct-specific. It was also found that human growth hormone had been accumulated in a concentration of several nanograms per milliliter (ng/ml) in the egg white. These and other findings have now led to completion of the present invention.

The possibility of oviduct-specific protein production has been proved by the fact that human growth hormone could be produced in transgenic birds by using, in accordance with the present invention, that region of the promoter for ovalbumin occurring in the egg white which causes oviduct-specific expression. As a result, it has become possible to efficiently accumulate desired proteins in eggs and, thus, the invention provides an excellent method for the accumulation of Fc portion-free useful proteins in eggs.

The invention is directed to a method of producing a useful protein, which comprises causing accumulation of the useful protein in eggs of a transgenic bird in which the protein is expressed through the use of an oviduct-specific expression promoter, and to a G0 transgenic chimera bird produced by such method as well as a G1 transgenic bird and offspring thereof.

Thus, the present invention provides a method of producing of a protein,

which comprises producing said protein into eggs laid by a female G1 transgenic bird, a female transgenic bird descended therefrom, or a female G0 transgenic chimera bird,

said female G1 transgenic bird being obtained through the following steps:

a) introducing a replication-defective retrovirus vector coding for the protein under the control of a promoter expressible in an oviduct-specific manner into fertile eggs of a bird,

b) allowing the eggs to hatch to give the G0 transgenic chimera bird and

c) mating one of the G0 transgenic chimera birds with another one of the Go transgenic chimera birds or with a wild type counterpart bird.

The invention also provides a G0 transgenic chimera bird obtained by introducing a replication-defective retrovirus vector coding for a protein under the control of a promoter expressible in an oviduct-specific manner into fertile eggs of a bird and allowing the egg to hatch, a G1 transgenic bird obtained by mating the G0 transgenic chimera bird with another G0 transgenic bird obtained in the same manner or with a wild type counterpart bird, or an offspring thereof.

### BRIEF DESCRIPTION OF THE DRAWINGS

FIG. 1 shows the structure of a vector construct, pLG0 G, derived from a replication-defective retrovirus vector. In the figure, ψ indicates the packaging signal sequence. GFP indicates the green fluorescent protein gene. OVApro indicates the ovalbumin promoter gene. hGH indicates the human growth hormone cDNA. 5′LTR and 3′LTR respectively indicate the long terminal repeats of MoMLV. Ampr indicates the ampicillin resistance gene.

FIG. 2 shows the hatchability levels in transgenic embryos. The hatchability levels are each shown in terms of percentage of hatches relative to the number of transgenic embryos.

FIG. 3 shows the results of analysis of genomic DNAs collected from hatched embryos by PCR. Molecular weight markers are shown under M. The numbers respectively indicate the G0 individuals after hatching. GFP indicates the green fluorescent protein gene.

FIG. 4 shows the transmission efficiencies of transgene to G1 . Each G0 No. indicates the number given to the relevant G0 . The transmission efficiency (%) is given in terms of the percentage of G1 individuals having the transgene among the G1 's tested.

FIG. 5 shows how the human growth hormone gene was expressed in a tissue-specific manner under the control of the ovalbumin promoter. M indicates a marker, B brain, H heart, L liver, S spleen, and O oviduct. As a negative control (NC), PCR was carried out without using any template and, as a positive control (PC), PCR was carried out using pLG0 G as a template.

FIG. 6 shows the results of measurements for human growth hormone concentration (ng/ml) in the white of eggs laid by G1 quails and a G0 chicken.

### DETAILED DESCRIPTION OF THE INVENTION

The G0 transgenic chimera bird and G1 transgenic bird according to the invention are characterized in that they are birds resulting from introduction of a foreign protein gene by means of a replication-defective retrovirus vector and can produce the protein in eggs thereof.

The method of producing the G0 transgenic chimera bird or G1 transgenic bird according to the invention may be, but is not limited to, that previously developed by the present inventors (Japanese Kokai Publication 2002-176880).

The gene to be introduced into the bird in accordance with the invention is not particularly restricted but desirably is a gene not derived from any retrovirus. The gene not derived from any retrovirus is not particularly restricted but includes those genes coding for proteins used as markers, for example green fluorescent protein (GFP), and those genes coding for useful proteins such as human growth hormone, erythropoietin (EPO) and antibodies, among others.

The gene to be introduced is inserted between the 5′ terminus and 3′ terminus of the provirus by the vector construct. For efficient accumulation of the product of this transgene in eggs, the promoter sequence of a gene expressed in an oviduct-specific manner is used. The term “promoter expressible in an oviduct-specific manner” as used herein means a gene sequence serving to regulate the expression of a protein contained in the egg white, and such promoter may be, but is not limited to, any of the promoters of the genes expressed in an oviduct-specific manner, such as the ovalbumin promoter. In particular, the chicken-derived ovalbumin promoter is judiciously used since its analysis has advanced and the expression-controlling region has been made clearer.

The replication-defective retrovirus vector to be used in the practice of the invention is not particularly restricted but may be any of those incapable of replication. Preferred, however, are those derived from the Moloney murine leukemia virus (MoMLV) and avian leukosis virus (ALV).

From the safety viewpoint, the retrovirus vector to be used as the vector for gene transfer should be one incapable of self replication as a result of the lack of one or more of gag, pol and env, which are necessary for replication.

The packaging cells can be produced by introducing the gag, pol and env genes and a plasmid coding for the retrovirus vector into appropriate cells. The method of introducing the retrovirus vector-encoding plasmid into GP 293 cells constitutively expressing gag and pol is not particularly restricted but includes, among others, the lipofection method, calcium phosphate method, and electroporation method. The envelope protein (env) to be used for efficiently infecting bird cells with the replication-defective retrovirus is desirably, but is not limited to, a vesicular stomatitis virus (VSV) envelope protein (VSV-G) pseudotype. The VSV-G protein-expressing GP 293 cells may be prepared by transfecting the GP 293 cells constitutively expressing gag and pol with the VSV-G protein expression vector and with the retrovirus vector-encoding plasmid simultaneously. This method is not a limited one, however.

The pseudotype virus produced by the packaging cells or the like is introduced into the embryo generally by the microinjection technique (Bosselam, R. A. et al. (1989), Science, 243, 533). The virus introduction can also be performed by lipofection or electroporation, for instance. The site of introduction is not limited to the embryo but the virus may be introduced into the blood vessel or heart after differentiation.

The method of producing the G0 transgenic chimera bird according to the invention is not particularly restricted but may comprise, for example, introducing a construct derived from a replication-defective retrovirus vector having a VSV-G protein-containing membrane by insertion of the above promoter and the gene to be introduced as inserted therein into the bird blastoderm, or postdifferentiation blood vessel or heart, followed by hatching the blastoderm. Usable as the method of hatching is, for example, the artificial eggshell method developed by the present inventors (Kamihira, M. et al. (1998), Develop. Growth Differ., 40, 449).

The birds to be used in the practice of the invention are not particularly restricted but include, among others, chickens, quails, turkeys, ducks and the like. Among them, chickens and quails are readily available and, when evaluated as egg-laying species, they are rich in fertility, and their safety has been established by long years of raising experience with them, hence are preferred.

The transgenic bird produced by the method of the present invention has the transgene introduced in a mosaic and chimeric manner in the somatic cell thereof, and the first generation thereof is referred to as “G0 transgenic chimera bird”, and the transgene-carrying birds descended therefrom are called G1 , G2, G3, ... transgenic birds.

By growing the G0 transgenic chimera bird to an adult and mating this with a nontransgenic bird (wild type bird) or another G0 transgenic chimera bird, it is possible to transmit the gene introduced into the G0 transgenic chimera bird to the descendant G1 transgenic birds. The success or failure in gene transmission may be determined by extracting DNA from blood or cells, for instance, from the G1 transgenic bird obtained and testing the DNA for gene transfer by the PCR method or hybridization method, for instance.

The oviduct-specific gene expression in the G0 transgenic chimera bird and in the G1 transgenic bird produced may be checked by collecting mRNA from the oviduct and some other tissue or blood and comparing the mRNAs by the RT-PCR method or Northern blot method, for instance.

In the practice of the invention, the protein production in eggs may be found either in the egg yolk or in the egg white. Preferred, however, is the protein production in the white. Presumably, protein accumulation in the white can be attained when a promoter expressed in an oviduct-specific manner is used.

In the case of a G1 transgenic bird for the expression of the human growth hormone gene under the control of the ovalbumin promoter, for instance, the content of human growth hormone in the egg white is preferably not less than 0.1 ng/ml, more preferably not less than 1 ng/ml. The method of assaying human growth hormone in the white is not particularly restricted but, for example, the commercial human growth hormone assaying kit hGH ELISA (product of Roche) and the like may be used.

It was shown that when the expression of a foreign protein is controlled using a promoter expressible in an oviduct-specific manner in accordance with the present invention, oviduct-specific foreign protein expression is possible and the foreign protein can be efficiently accumulated in the egg. Furthermore, as shown, the G0 transgenic chimera quail can transmit the transgene to G1 quails or, in other words, it is also possible to produce G1 transgenic quails. Thus, in producing a useful protein, it is now possible to efficiently accumulate the protein in the egg. By recovering the same, it is possible to produce the useful protein at low cost.

### BEST MODES FOR CARRYING OUT THE INVENTION

The following examples illustrate the present invention in detail. These examples are, however, by no means limitative of the scope of the invention.

### Example 1

A retrovirus vector for the expression of human growth hormone using the ovalbumin promoter was constructed.

1. Extraction of Genomic DNA from Chicken Fibroblasts

For chicken genome extraction, a fertile chicken egg was first incubated for 10 days. The chicken embryo was then taken out and cut to pieces with scissors. The cut embryo was treated with a 0.5 g/l solution of collagenase (product of Wako Pure Chemical Industries) for 5 to 10 minutes. After washing with two portions of PBS, the cut embryo was further finely divided by pipetting and cultured on DMEM (Dulbecco's modified Eagle's medium; product of Sigma) at 38.5° C. for 2 days. Cells were recovered, washed with PBS, and the pellet obtained was suspended in 1 ml of TNE (Tris (pH 8.0), 150 mM NaCl, 10 mM EDTA). After addition of 0.1 ml of 10% SDS and 0.05 ml of 20 mg/ml Proteinase K, the suspension was incubated at 55° C. for 1 hour and then incubated overnight at 37° C. This solution was subjected to several times of phenol-chloroform treatment, followed by ethanol precipitation for genome extraction.

2. Construction of a Chicken Genomic DNA Phage Library

The chicken genome prepared was partially digested with MboI, followed by sucrose density gradient centrifugation, and fragments about 23 Kb in size were recovered and joined to the phage vector EMBL3 (product of STRATAGENE), followed by packaging using the packaging kit Gigapack III Gold (product of STRATAGENE) according to the manual attached thereto to give a phage library.

3. Plaque Hybridization The library was diluted and used to infect host cells, namely Escherichia coli LE 392 cells, the cells were suspended in top agar, and the suspension was layered on an LB plate and incubated overnight at 37° C. for plaque formation. The nylon membrane filter Hybond-N (product of Amersham BioScience) was placed on the plaque-bearing plate in order to transfer phages to the filter. The membrane after transfer was denatured by immersion in a denaturing solution (1.5 M NaCl, 0.5 M NaOH), and then neutralized by immersion in a neutralizing solution (1.5 M NaCl, 0.5 M Tris-HCl (pH 7.5)). The membrane was then washed with 2 ×SSC (1.75% NaCl, 0.88% C₆H₅O₇Na₃ ₂H₂O (pH 7.0)), and baked at 80° C. for 2 hours. A probe for plaque hybridization was prepared by carrying out PCR using, as primers, two chemically synthesized oligonucleotides adapted for amplifying about 1 Kb just before the TATA box of the ovalbumin gene, namely (OVApro5) 5′-ctgtggtgtagacatccagca-3′ (SEQ ID NO:1) and (OVApro3) 5′-tttgacctttgacgccatag-3′ (SEQ ID NO:2). The probe of the PCR product obtained was labeled using. the random primer labeling kit (BcaBEST Labeling Kit, product of TaKaRa). About 100,000 plaques on the membrane carrying the phages transferred were analyzed by plaque hybridization using that probe, and a positive plaque (15′-A) was cloned.

4. Analysis of the Cloned Phage DNA

DNA was extracted from this phage and analyzed, upon which it was confirmed that it contained a 17-Kb fragment. For further confirmation, primers were chemically synthesized for the 3.5 Kb region upstream of the TATA box and said to be necessary for oviduct-specific expression of the ovalbumin exon 1 (OVA-3.5kb5; 5′-cacatccaaaggagcttgacc-3′ (SEQ ID NO:3), OVA-3.5kb3; 5′-cagttggaaggttacctggga-3′ (SEQ ID NO:4)). Using these primers, or OVApro5 and OVApro3, PCR was carried out with the 15′-A phage DNA as the template. Amplification of a segment of about 0.6 Kb or 1.1 Kb was confirmed, respectively. Then, primers capable of annealing with each end of the EMBL3 vector were chemically synthesized (5′-actcgtgaaaggtaggcggatctg-3′ (SEQ ID NO:5) and 5′-cgtccgagaataacgagtggatctg-3′ (SEQ ID NO:6)). Using these primers, the base sequences of both ends of the insert DNA were determined. As a result, it could be confirmed that a segment starting with the 3847 bp upstream of the exon 1 of the ovalbumin gene had been cloned in the right arm of EMBL3. However, the base sequence determination from the left arm of EMBL3 failed to specify the relevant gene. It was thus thought that two or more inserts had been cloned in the 15′-A clone. Therefore, an attempt was made to confirm, by using a restriction enzyme and the PCR technique, whether the phage DNA contained the ovalbumin initiation codon necessary in utilizing the ovalbumin promoter. For confirming whether the KpnI site occurring at a site downstream from the ovalbumin initiation codon had been cloned, the phage DNA was cleaved with KpnI, whereupon a 7 Kb fragment was confirmed. A primer (OVA3060), 5′-cattggcatggtggacttt-3′ (SEQ ID NO:7), was chemically synthesized for about 60 bp downstream from the initiation codon. Using OVApro5 and OVA3060, PCR was carried out with the 15′-A phage DNA as the template. As a result, a DNA of about 3 Kb was amplified. From the results of treatment with the restriction enzyme KpnI and PCR, it was confirmed that the 15′-A phage DNA contained the ovalbumin initiation codon and 1.9 Kb downstream therefrom. To sum up, it was confirmed that a total length of about 7.4 Kb, including a segment from 3.8 Kb upstream of the exon 1 of the ovalbumin gene to the ovalbumin initiation codon and including the restriction enzyme KpnI site at least about 1.9 Kb downstream from the initiation codon, had been cloned in the 15′-A phage DNA.

5. Human Growth Hormone cDNA Preparation

A genomic DNA sequence of human growth hormone was prepared from HUVECs (Human Umbilical Vein Endothelial Cells). As for the preparation method, HUVECs were first cultured in TCM 199 medium containing 10% FCS and 30 μg/ml Endothelial Cell Growth Supplement (ECGS; product of Becton Dickinson Labware). The genome was prepared from the cells recovered after cultivation using MagExtractor Genome (product of Toyobo). PCR was carried out using chemically synthesized primers 5′-cagctcaaggatcccaaggccc-3′ (SEQ ID NO:8) and 5′-ggacacctagtcagacaaaatgatgcaac-3′ (SEQ ID NO:9), with the above-prepared genome as the template. With the PCR product as the template, PCR was again carried out using chemically synthesized primers 5′-atactcgaggttcaccatggctacaggtaagcgcc-3′ (SEQ ID NO;10; the underlined portion being an XhoI restriction enzyme site) and 5′-aatctcgagacgcgtggacacctagtcagacaaa-3′ (SEQ ID NO:11; the underlined portion being an XhoI-MluI restriction enzyme site). The fragment amplified by PCR was cleaved with XhoI and the resulting fragment was inserted into the plasmid pZeoSV2(+) (product of Invitrogen) at the XhoI site. Thus was constructed a human growth hormone genomic gene-containing plasmid, pZeogGH. Since this chromosome-derived human growth hormone gene contained introns, an attempt was made to prepare an intron-free cDNA. The plasmid pZeogGH constructed was transfected into CHO cells (obtained from CELL BANK of RIKEN (The Institute of Physical and Chemical Research)) by the lipofection technique. The transfected CHO cells were cultured on an F12 medium and, thereafter, mRNA was recovered using an mRNA isolation kit (product of Roche). Using the mRNA obtained and chemically synthesized primers 5′-atggctacaggctcccggacgtccct-3′ (SEQ ID NO:12) and 5′-cagctagaagccacagctgccctccacag-3′ (SEQ ID NO:13), RT-PCR was carried out using RT-PCR Beads (product of Amersham Bioscience). The RT-PCR product obtained was subjected to PCR using chemically synthesized primers 5′-atactcgagaccatggctacaggctcccg-3′ (SEQ ID NO:14; the underlined portion being an XhoI restriction enzyme site) and 5′-aatctcgagacgcgtagctagaagccacagctgc-3′ (SEQ ID NO:15; the underlined portion being an XhoI-MluI restriction enzyme site) to give a human growth hormone cDNA. This cDNA was treated with XhoI and then inserted into pBluescript SK+ (product of STRATAGENE) at the XhoI site. A plasmid, pBluecGH, was thus constructed.

6. Construction of an Ovalbumin Promoter-Dependent Human Growth Hormone Expression Retrovirus Vector

A plasmid, PLG0 G, coding for an ovalbumin promoter-dependent human growth hormone expression retrovirus vector was constructed in the following manner.

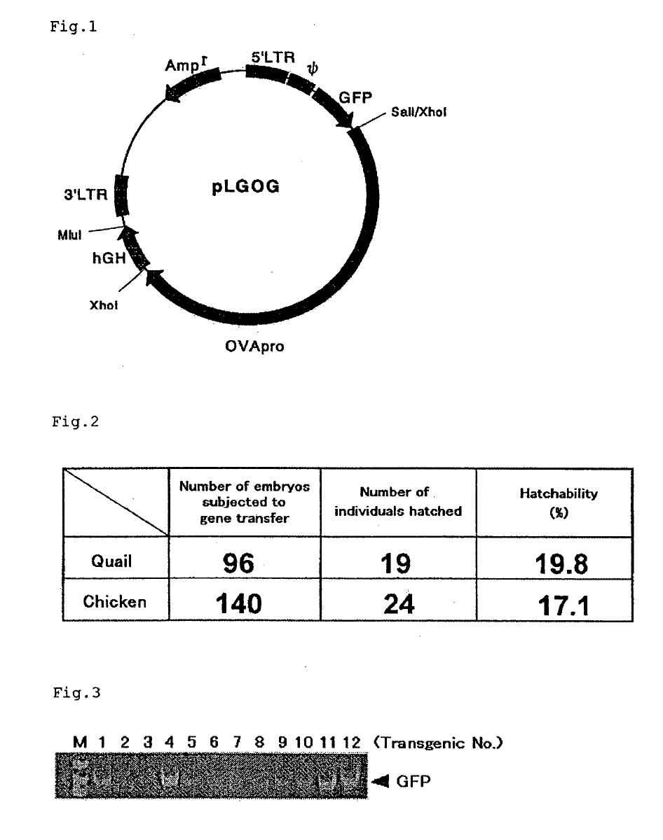

First, a 7-Kb fragment was excised from the phage DNA in which the ovalbumin promoter had been cloned by cleavage with SalI-KpnI and inserted into pUC19 at the SalI-KpnI site thereof, whereby a plasmid, p19OVA-3.5Kb, was constructed. For adding an XhoI restriction enzyme site just before the ovalbumin initiation codon, 5′-atcaagcttctcctagactacatgaccccata- 3′ (SEQ ID NO:16; the underlined portion being a HindIII restriction enzyme site) and 5′-atcctcgagttgtctagagcaaacagcaga-3′ (SEQ ID NO:17; the underlined portion being an XhoI restriction enzyme site) were chemically synthesized. Amplification was effected by carrying out PCR using p19OVA-3.5Kb as the template. The fragment thus amplified was treated with HpaI and XhoI, and a 120-bp fragment was recovered. Furthermore, for the joining of the green fluorescent protein (GFP) gene, primers were chemically synthesized (5′-atgctcgaggttcaccatgagcaagggcgagg-3′ (SEQ ID NO:18; the underlined portion being an XhoI restriction enzyme site) and 5′-atcggtaccgcatgcacgcgtcgatccagacatgataagata-3′ (SEQ ID NO:19; the underlined portion being a KpnI-SphI-MluI restriction enzyme site)). Using these primers, PCR was carried out with the plasmid PGREEN LANTERN-1 (product of GIBCO) as the template. The fragment obtained was treated with XhoI and KpnI. These two fragments were simultaneously inserted into pl9OVA-3.5Kb at the HpaI-KpnI site to construct pOVA-GFP. A fragment excised from the human growth hormone genomic DNA-containing plasmid pZeogGH with XhoI and MluI was inserted into pOVA-GFP at the XhoI-MluI site to construct a plasmid, pOVA-gGH. PCR was carried out using the retrovirus vector pLGRN (Japanese Kokai Publication 2002-176880) as the template, together with chemically synthesized primers 5′-catacgcgttcttcggaccctgcattc-3′ (SEQ ID NO:20; the underlined portion being an MluI restriction enzyme site) and 5′-tgcggtattttctccttacgcatc-3′ (SEQ ID NO:21), and using Pyrobest DNA Polymerase capable of giving blunt-ended PCR products. The fragment thus amplified was treated with XbaI and inserted into pBluescript SK(+) at the SmaI-XbaI site to construct a plasmid, pBlue3′ LTRMluXba. An XhoI-XbaI fragment was excised from this plasmid pBlue3′LTRMluXba and inserted into pLGRN at the XhoI-XbaI site to construct a plasmid, pLG. A fragment was excised from the ovalbumin promoter- and human growth hormone genomic DNA-containing plasmid pOVA-gGH with SalI and MluI and inserted into pLG at the XhoI-MluI site to construct pLG0 gG. A fragment was excised from the human growth hormone cDNA-containing plasmid pBluecGH with XhoI and MluI and inserted into pLG0 gG at the XhoI-MluI site, whereby a plasmid, pLG0 G, coding for a replication-defective retrovirus vector allowing the expression of human growth hormone under the control of the ovalbumin promoter was constructed. A map of this plasmid is shown in FIG. 1.

### Example 2

Preparation of a viral vector for the expression of human growth hormone using the ovalbumin promoter

For preparing a retrovirus from the vector construct pLG0 G constructed in Example 1, GP 293 cells (product of Clontech) were cultured on a culture dish with a diameter of 100 mm using DMEM. GP 293 cells were re-sown on the previous day to 90% confluence, and then transfected with 11 μg of the plasmid pLG0 G and 11 pg of the plasmid pVSV-G (product of Clontech) by the lipofection technique. After 48 hours, the supernatant was deprived of contaminants by passing through a 0.45-μm cellulose acetate filter (product of Advantech) and then ultra-centrifuged at 25,000 rpm and at 4° C. for 1.5 hours. The supernatant was discarded, and the pellet was dissolved in TNE (50 mM Tris-HCl (pH 7.8), 130 mM NaCl, 1 mM EDTA) to give a virus solution. This virus solution was diluted with DMEM containing 10 μg/ml of polybrene (product of Sigma). The medium used for cultivating GP 293 cells on a 24-well microplate was exchanged for this virus dilution in polybrene-containing DMEM. After cultivation for growth, the cells were recovered and diluted with DMEM to 13.3 cells/ml, and the dilution was distributed in 150-μl portions into the wells of a 96-well microplate. Two weeks later, the colonies formed were transferred to 24-well microplates. From among them, the one showing high-titer was selected and used as packaging cells. The packaging cells were cultured and transfected with the pVSV-G plasmid by the lipofection technique. After 48 hours, the supernatant was deprived of contaminants by passing through a 0.45-μm cellulose acetate filer (product of Advantech) and then ultra-centrifuged at 25,000 rpm and at 4° C. for 1.5 hours. The supernatant was discarded, and the sediment was dissolved in TNE (50 mM Tris-HCl (pH 7.8), 130 mM NaCl, 1 mM EDTA) to give a virus solution. The thus-obtained high-titer virus solution had a titer of 1×10⁸ to 8×10⁸ cfu/ml.

The virus titer measurement was carried out in the following manner. On the day before the measurement day, a 24-well dish was sowed with 1.5×10⁴ NIH 3T3 cells (obtained from American Type Culture Collection), which were then cultured. The cell culture medium was replaced with 1 ml of a virus solution 10² to 10⁶-times diluted with DMEM containing 10 μg/ml polybrene and, after 48 hours, the proportion of cells expressing GFP (green fluorescent protein) was determined under a fluorescence microscope. The titer was calculated according to the following calculation formula:

(Virus titer)=(number of cells)×(dilution ratio)×(expression proportion) (cfu/ml)

### Example 3

Retrovirus Injection into the Quail Embryo and Embryo Culture

Fertile WE strain quail eggs (Nippon Institute for Biological Science) were used. The eggshell of each fertile egg just after oviposition was sterilized with 70% ethanol, and a circular portion thereof, 2 cm in diameter, at the sharper end of the egg was cut off with a diamond cutter (MINIMO 7C710; product of Minitor) to expose the embryo. A glass tube (GD-1; product of Narishige) was worked on a micropipet maker (PC-10; product of Narishige), the virus solution prepared in Example 2 was placed in the glass tube, the needle made by breaking the tip to attain an outside diameter of about 20 μm was stabbed into the embryo under observation of the blastoderm under a stereoscopic microscope, and 2 μl of the solution was injected into the middle of the subgerminal cavity using a microinjector (Transjector 5246; product of Eppendorf). The eggshell was filled with the egg white to the cut end, and the opening was covered with a PTFE film (MilliWrap; product of Millipore) and a polyvinylidene chloride wrap film (Saran Wrap; product of Asahi Kasei) using the egg white as a paste. This virus-injected fertile egg was incubated in an incubator (Showa Furanki model P-008) with a built-in automatic egg rotator while turning the egg by 90° at 15-minute intervals. Two days after the start of incubation, an S-size chicken egg was sterilized with 70% ethanol, a circular portion, 3.5 cm in diameter, at the blunter end thereof was cut off with a diamond cutter, the contents were removed, the white and yolk of the quail egg injected with the virus were transferred to the chicken eggshell, 0.5 ml of a calcium lactate solution suspended in egg white at a concentration of 50 mg/ml was added, and the opening was covered with a polyvinylidene chloride wrap film (Saran Wrap; product of Asahi Kasei) using the egg white as a paste. The embryo transferred to the S-size chicken egg was incubated in an incubator with a built-in automatic egg rotator while turning the egg by 30° at 15-minute intervals.

### Example 4

Retrovirus Injection into the Chicken Embryo and Embryo Culture

Fertile chicken eggs (Nippon Institute for Biological Science) were used. The eggshell of each egg just after oviposition was sterilized with 70% ethanol, and a circular portion thereof, 3.5 cm in diameter, at the sharper end of the egg was cut off with a diamond cutter (MINIMO 7C710; product of Minitor) to expose the embryo. A glass tube (GD-1; product of Narishige) was worked on a micropipet maker (PC-10; product of Narishige), the virus solution prepared in Example 2 was placed in the glass tube, the needle made by breaking the tip to attain an outside diameter of about 20 μm was stabbed into the embryo under observation of the blastoderm under a stereoscopic microscope, and 2 μl of the solution was injected into the middle of the subgerminal cavity using a microinjector (Transjector 5246; product of Eppendorf). The eggshell was filled with the egg white to the cut end, and the opening was covered with a PTFE film (MilliWrap; product of Millipore) and a polyvinylidene chloride wrap film (Saran Wrap; product of Asahi Kasei) using the egg white as a paste. This virus-injected fertile egg was incubated in an incubator (Showa Furanki model P-008) with a built-in automatic egg rotator while turning the egg by 900 at 15-minute intervals. Two days after the start of incubation, an LL-size chicken egg was sterilized with 70% ethanol, a circular portion, 4.5 cm in diameter, at the blunter end thereof was cut off with a diamond cutter, the contents were removed, the white and yolk of the chicken egg were transferred to the LL-size eggshell, 0.5 ml of a calcium lactate solution suspended in egg white at a concentration of 50 mg/ml was added, and the opening was covered with a polyvinylidene chloride wrap film (Saran Wrap; product of Asahi Kasei) using the egg white as a paste. The embryo transferred to the LL-size chicken egg was incubated in an incubator with a built-in automatic egg rotator while turning the egg by 30° at 15-minute intervals.

### Example 5

Hatchability of Transgenic Bird Embryos

A total of 96 quail embryos subjected to replication-defective retrovirus vector introduction by two runs of the procedure for virus introduction into the embryo, and a total of 140 chicken embryos subjected to replication-defective retrovirus vector introduction by four runs of the procedure for virus introduction into the embryo were hatched by the method shown in Example 4. The transgenic bird embryos could be hatched with an average hatchability of 19.8% in quails, and 17.1% in chickens. The hatchability data for transgenic quail embryos are shown in FIG. 2.

### Example 6

PCR Analysis of the Genomic DNA Collected from Hatched Embryos

The chorioallantoic membrane of the eggshell of each hatched chicken or quail was collected, and a transgenic chimera bird genomic DNA was prepared using MagExtractor Genome (product of Toyobo). The genomic DNA prepared was subjected to PCR using chemically synthesized primers amplifiable within GFP by PCR, namely 5′-tggtgaatagaatcgagctgaagggcatt-3′ (SEQ ID NO:22) and 5′-aactccagcaggaccatgtggtctctctt-3′ (SEQ ID NO:23). The results are shown in FIG. 3. As is evident from the results, the transgene could be detected in all quail individuals tested. In chickens, the introduction efficiency was 70%. It was thus confirmed that the present inventors could have produced G0 transgenic birds with high efficiency.

### Example 7

Producing of G1 Transgenic Quails and Transmission Efficiency

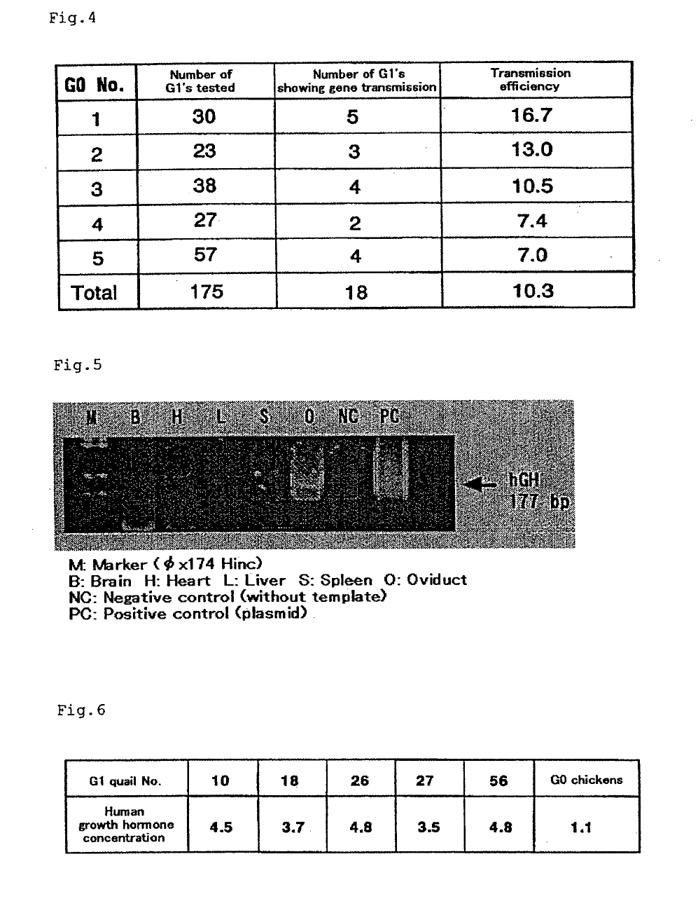

Since the embryo of each G0 transgenic chimera bird is already composed of a number of cells at the time of gene transfer, it is presumable that cells with the gene in question introduced therein and cells free of the gene form a mosaic. Thus, an attempt was made to produce G1 transgenic quails. G0 transgenic chimera quails were naturally mated with non-transgenic quails, and the G1 quails thus born were tested for transgene transmission efficiency by the test method applied to the transgenic chimera quails in Example 6. As a result, a total of 175 nestlings born from 5 G0 transgenic chimera quails were analyzed, and the transgene was detected in 18 nestlings, hence the transmission efficiency was about 10%. The data for the efficiency of this transmission from G0 transgenic chimera quails to G1 quails are shown in FIG. 4.

### Example 8

Analysis of Expression of Human Growth Hormone in G1 Transgenic Quails

Whether the human growth hormone expression was oviduct-specific or not was checked in the G1 transgenic quails produced. Each tissue of each mature female was collected, the mRNA was obtained using an mRNA isolation kit (product of Roche), and RT-PCT was carried out using the chemically synthesized primers, 5′-agtattcattcctgcagaacccccagacc-3′ (SEQ ID NO:24) and 5′-ctgttggcgaagacactcctgaggaactg-3′ (SEQ ID No:25) made from the mRNA obtained, and using RT-PCR Beads (product of Amersham Bioscience). The results are shown in FIG. 5. As the results indicate, the human growth hormone gene introduced was found to have been expressed in the oviduct while no expression thereof was observed in other tissues. It was thus confirmed that the ovalbumin promoter introduced functioned in an oviduct-specific manner.

### Example 9

Analysis for Human Growth Hormone Accumulation in the egg white of G1 Transgenic Quails and of G0 Transgenic Chimera Chickens

Eggs laid by each female G1 transgenic quail after maturation were collected and assayed for the human growth hormone concentration in egg white by the ELISA technique. For assaying, the commercial human growth hormone assaying kit hGH ELISA (product of Roche) was used. With this kit, it is possible to assay several tens of picograms/ml of human growth hormone. The results of measurements of human growth hormone concentrations in the egg white by ELISA are shown in FIG. 6. As a result, it was revealed that human growth hormone had been produced at a concentration of several nanograms per milliliter (ng/ml) in the egg white in every G1 transgenic quail. Furthermore, as a result of assaying of the egg white of female G0 transgenic chimera chickens, the hormone concentration was found to be 1.1 ng/ml.

1. A method of producing a protein,
which comprises producing said protein into eggs laid by a female G1 transgenic bird, a female transgenic bird descended therefrom, or a female G0 transgenic chimera bird,
said female G1 transgenic bird being obtained through the following steps:
a) introducing a replication-defective retrovirus vector coding for the protein under the control of a promoter expressible in an oviduct-specific manner into fertile eggs of a bird,
b) allowing the eggs to hatch to give the G0 transgenic chimera bird and
c) mating one of the G0 transgenic chimera birds with another one of the G0 transgenic chimera birds or with a wild type counterpart bird.

2. The method according to claim 1,
wherein the site of introduction of the replication-defective retrovirus vector is the fertile egg embryo, blood vessel, or heart.

3. The method according to claim 1,
wherein the site of introduction of the replication-defective retrovirus vector is the fertile egg embryo.

4. The method according to claim 1,
wherein the introduction of the replication-defective retrovirus vector is carried out by the microinjection, lipofection, or electroporation technique.

5. The method according to claim 1,
wherein the introduction of the replication-defective retrovirus vector is carried out by the microinjection technique.

6. The method according to claim 1,
wherein the protein production is realized in the white of eggs produced by the female G0 transgenic chimera bird.

7. The method according to claim 1,
wherein the protein production is realized in the white of eggs produced by the female G1 transgenic bird or an offspring thereof.

8. The method according to claim 1,
wherein the promoter expressible in an oviduct- specific manner is the chicken ovalbumin promoter.

9. The method according to claim 1,
wherein the protein is human growth hormone.

10. A G0 transgenic chimera bird obtained by introducing a replication-defective retrovirus vector coding for a protein under the control of a promoter expressible in an oviduct-specific manner into fertile eggs of a bird and allowing the egg to hatch.

11. A transgenic bird,
which is a G1 transgenic bird obtained by mating the transgenic bird according to claim 10, namely the G0 transgenic chimera bird, with another G0 transgenic bird obtained in the same manner or with a wild type counterpart bird, or an offspring thereof.

12. The transgenic bird according to claim 10,
which is a chicken or quail.

13. The transgenic bird according to claim 10,
wherein the protein is human growth hormone.

14. The transgenic bird according to claim 13,
which produces human growth hormone in eggs and, which is a female transgenic chimera bird or a female transgenic bird.

15. The transgenic bird according to claim 14,
wherein human growth hormone is contained in the white of eggs thereof in an amount of not less than 0.1 ng/ml.

16. The transgenic bird according to claim 14,
wherein human growth hormone is contained in the white of eggs thereof in an amount of not less than 1 ng/ml.


SEQUENCE LISTING (21 sequences)

SEQ ID NO: 1 - DNA, 21 bp, Artificial Sequence
  Designed sequence of a 5′-primer used for PCR amplification of  TATAbox 1kb upstream region of the ovalbumin promoter
ctgtggtgtagacatccagca

SEQ ID NO: 2 - DNA, 20 bp, Artificial Sequence
  Designed sequence of a 3′-primer used for PCR amplification of  TATAbox 1kb upstream region of the ovalbumin promoter
tttgacctttgacgccatag

SEQ ID NO: 3 - DNA, 21 bp, Artificial Sequence
  Designed sequence of a 5′-primer used for PCR amplification of  TATAbox 3.5kb upstream region of the ovalbumin promoter
cacatccaaaggagcttgacc

SEQ ID NO: 4 - DNA, 21 bp, Artificial Sequence
  Designed sequence of a 3′-primer used for PCR amplification of  TATAbox 1kb upstream region of the ovalbumin promoter
cagttggaaggttacctggga

SEQ ID NO: 5 - DNA, 24 bp, Artificial Sequence
  Designed sequence of a 5′-primer used for sequence of EMBL3 insert DNA
actcgtgaaaggtaggcggatctg

SEQ ID NO: 6 - DNA, 25 bp, Artificial Sequence
  Designed sequence of a 3′-primer used for sequence of EMBL3 insert  DNA
cgtccgagaataacgagtggatctg

SEQ ID NO: 7 - DNA, 19 bp, Artificial Sequence
  Designed sequence of a 3′-primer used for PCR amplification that was located 60bp downstream of the ovalbumin start codon
cattggcatggtggacttt

SEQ ID NO: 8 - DNA, 22 bp, Artificial Sequence
  Designed sequence of a 5′-primer used for PCR amplification of  genomic human growth hormone gene
cagctcaaggatcccaaggccc

SEQ ID NO: 9 - DNA, 29 bp, Artificial Sequence
  Designed sequence of a 3′-primer used for PCR amplification of genomic human growth hormone gene
ggacacctagtcagacaaaatgatgcaac

SEQ ID NO: 10 - DNA, 35 bp, Artificial Sequence
  Designed sequence of a 5′-primer incorporating XhoI recognition site at the 5′ terminal used for PCR amplification of amplified human growth hormone gene
atactcgaggttcaccatggctacaggtaagcgcc

SEQ ID NO: 11 - DNA, 34 bp, Artificial Sequence
  Designed sequence of a 3′-primer incorporating XhoI and MluI recognition site at th 5′ terminal used for PCR amplification of  amplified human growth hormone gene
aatctcgagacgcgtggacacctagtcagacaaa

SEQ ID NO: 12 - DNA, 26 bp, Artificial Sequence
  Designed sequence of a 5′-primer used for RT-PCR amplification of cloned human growth hormone mRNA
atggctacaggctcccggacgtccct

SEQ ID NO: 13 - DNA, 29 bp, Artificial Sequence
  Designed sequence of a 3′-primer used for RT-PCR amplification of cloned human growth hormone mRNA
cagctagaagccacagctgccctccacag

SEQ ID NO: 14 - DNA, 29 bp, Artificial Sequence
  Designed sequence of a 5′-primer incorporating XhoI recognition site at th 5′ terminal used for PCR amplification of amplified human growth hormone cDNA
atactcgagaccatggctacaggctcccg

SEQ ID NO: 15 - DNA, 34 bp, Artificial Sequence
  Designed sequence of a 5′-primer incorporating XhoI and MluI recognition site at the 5′ terminal used for PCR amplification of amplified human growth hormone cDNA
aatctcgagacgcgtagctagaagccacagctgc

SEQ ID NO: 16 - DNA, 32 bp, Artificial Sequence
  Designed sequence of a 5′-primer incorporating HindIII recognition site at the 5′ terminal used for PCR amplification of ovalbumin promoter
atcaagcttctcctagactacatgaccccata

SEQ ID NO: 17 - DNA, 30 bp, Artificial Sequence
  Designed sequence of a 3′-primer incorporating XhoI recognition site at the 5′ terminal used for PCR amplification of ovalbumin promoter
atcctcgagttgtctagagcaaacagcaga

SEQ ID NO: 18 - DNA, 32 bp, Artificial Sequence
  Designed sequence of a 5′-primer incorporating XhoI recognition site at the 5′ terminal used for PCR amplification of GFP
atgctcgaggttcaccatgagcaagggcgagg

SEQ ID NO: 19 - DNA, 42 bp, Artificial Sequence
  Designed sequence of a 3′-primer incorporating KpnI,SphI and MluI recognition site at the 5′ terminal used for PCR amplification of GFP
atcggtaccgcatgcacgcgtcgatccagacatgataagata

SEQ ID NO: 20 - DNA, 27 bp, Artificial Sequence
  Designed sequence of a 5′-primer incorporating MluI recognition site at the 5′ terminal used for PCR amplification of 3′LTR
catacgcgttcttcggaccctgcattc

SEQ ID NO: 21 - DNA, 24 bp, Artificial Sequence
  Designed sequence of a 5′-primer used for PCR amplification of 3′LTR
tgcggtattttctccttacgcatc